(19) United States
(12) Patent Application Publication
(10) Pub. No.: US 2022/0048027 A1
(43) Pub. Date: Feb. 17, 2022

(54) SAMPLE HANDLING DEVICE

(71) Applicant: MAGNASENSE TECHNOLOGIES OY, VANTAA (FI)

(72) Inventors: Ernest M. BATE, Kent (GB); Tom HAYDON, East Sussex (GB); Mika LAITINEN, VANTAA (FI)

(73) Assignee: MAGNASENSE TECHNOLOGIES OY, VANTAA (FI)

(21) Appl. No.: 17/429,188

(22) PCT Filed: Feb. 12, 2020
(86) PCT No.: PCT/FI2020/050087

§ 371 Date: Aug. 6, 2021

(30) Foreign Application Priority Data
Feb. 12, 2019 (FI) 20195102

(51) Int. Cl. B01L 3/00; A61B 5/15; A61B 5/157

(52) U.S. Cl. CPC B01L 3/50273; B01L 3/502753; A61B 5/150229; A61B 5/150755; A61B 5/157; B01L 2400/0478; B01L 2200/0689; B01L 2200/10; B01L 2300/0681; B01L 2400/0406; B01L 2200/028

(57) ABSTRACT

A sample handling device includes a reservoir for holding a fluid medium. A channel system used in connection with the reservoir includes a dilution portion for a sample to be analyzed with a measurement device. The sample is arranged to be transferred from the dilution portion to the measurement device by the fluid medium. A set of capillary channels in the dilution portion is arranged to be filled by capillary action to collect an established quantity of the sample to be diluted by the fluid medium. A pump transfers the fluid medium from the reservoir to the channel system. The pump includes at least one plunger, a seal separating the reservoir and the channel system and a delivery system of potential energy including a compressible element configured to provide repeatable transfer of the fluid medium from the reservoir to the channel system.

### CROSS-REFERENCE TO RELATED APPLICATIONS

This application is a U.S. National Stage of International Patent Application No: PCT/FI2020/050087 filed on Feb. 12, 2020, which claims benefit of Finland Patent Application No. FI 20195102 filed on Feb. 12, 2019.

### FIELD OF THE INVENTION

The invention relates to a sample handling device. More specifically, the present invention particularly relates to a sample handling device useful for performing blood tests in quick and simple ways.

### BACKGROUND OF THE INVENTION

Many Point of Care (POC) instruments make health-related measurements from a single drop of blood. A common example of such an instrument is the blood glucose meter used by people with diabetes.

Many POC measurements made from blood must be conducted on plasma or serum, which necessitates the removal of red blood cells (RBCs) from the whole blood sample. Trying to measure the same analytes in whole blood risks significant errors in the results because of the large natural variation in the proportion of RBCs in whole blood, which is even more variable for persons who are dehydrated or ill.

RBCs can easily be separated from whole blood by using conventional methods such as centrifugation and sedimentation. However these conventional methods require separate instruments which are very difficult to use on small volumes of whole blood. Thus, these conventional methods are not well suited to POC instruments.

Most RBC separation methods which currently exist for POC instruments are based on filtration and on microfluidics, but acoustic waves, dielectrophoresis and sedimentation have also been proposed. Examples of the materials and methods that have been used are given in several papers as cited below. Filtration has been employed with membranes, micropillars, microbeads, composite materials and papers. Microfluidic methods have included fractionation, inertial effects and bifurcation effects. Many of these methods are discussed and compared in the following papers:

H Shimizu et al, “Whole Blood Analysis Using Microfluidic Plasma Separation and Enzyme-Linked Immunosorbent Assay Devices”, Analytical Methods, 2016, DOI: 10.1039/C6AY01779G.

W S Mielczarek et al, “Microfluidic blood plasma separation for medical diagnostics: is it worth it?”, Lab Chip, 2016, 16, 3441, DOI:10.1039/C6LC00833J

S Mukherjee et al, “Plasma Separation from Blood: The ‘Lab-on-a-chip’ Approach”, Critical Reviews in Biomedical Engineering, January 2009, DOI: 10.1615/CritRevBiomedEng.v37.i6.40

H W Hou et al, “Microfluidic Devices for Blood Fractionation”, Micromachines, 2011, 2, 319-343, DOI: 10.3390/MI2030319

Jun Ho Sun et al, “Hemolysis-free blood plasma separation”, Lab Chip, 2014, 14, 2287-2292, DOI:10.1039/c41c00149d Several patents describe materials and devices to accomplish RBC removal and POC measurements. U.S. Pat. Nos. 4,816,224, 5,186,843 and 5,240,862 deal with materials and devices to separate RBCs from whole blood. U.S. Pat. Nos. 4,980,297, 5,135,719, 5,064,541, 5,139,685, 6,296,126B1, 6,197,598B1, 6,391,265B1, 7,279,136 and EP131553 and EP1096254B1 describe devices and methods for separating plasma from whole blood and integrating these devices with various detection methods and POC instruments.

All these prior art methods suffer from a variety of problems, including low efficiencies of retention and a tendency to leak RBCs, slow operating time and a requirement for more blood than is normally available from a finger prick. Many of the prior art systems are unable to provide free plasma that is capable of being used in a dilution and measurement system.

The prior art includes a description of materials that are well suited to separating RBCs quickly and without the need for increased pressure. For example, U.S. Pat. No. 4,753,776 deals with glass fiber filter papers that separate plasma from RBCs, using only capillary force, in forms that operate with and without the use of agglutinins.

The prior art also includes micro-machined or micro-fluidic devices. For example, U.S. Pat. No. 6,296,126B1 uses wedge shaped cutouts to facilitate removal of liquid from the matrix. These micro-fluidic devices usually give very low recoveries of plasma, however, as discussed in the H Shimizu et al 2016 paper.

Other prior art of interest includes U.S. Pat. No. 2011/0041591A1 which describes a system which attempts to overcome some of the existing problems. It collects the filtered plasma in a matrix by capillary action and then ejects the plasma by applying force to squeeze the plasma out of the matrix.

U.S. Pat. No. 2015/0182156A1 describes a test device which first dilutes a blood sample and then forces it through a filter. This system cannot give accurate results for some tests, however, unless hematocrit correction is used.

U.S. Pat. No. 7,544,324B2 describes devices for sample collection, fluid storage, mixing and analysis. This prior art addresses ease of use but does not perform RBC separation.

To enable quantitative measurements it is necessary to meter the volume of plasma which has been separated and to mix it with diluent in a repeatable manner which is not influenced by how the user operates the test. In some existing systems the dilution step is subject to variation depending on how fast or hard the user presses an actuator such as a syringe plunger. Some methods to reduce this variation are described in U.S. Pat. No. 2015/0182156A1.

Among all the existing art, there is no system that can quickly and simply separate and measure plasma from a single drop of blood and dilute the measured amount of it in a controlled manner such that the resulting fluid can be used for a health-related measurement. The invention disclosed addresses this need.

### SUMMARY OF THE INVENTION

An object of this invention is to provide a sample handling device to collect, meter, dilute and deliver the sample from the device to a measurement system, in a quick, simple and reproducible way that is also suitable for quantitative measurements. The sample handling device according to the present invention is characterized by a reservoir to hold a fluid medium; a channel system arranged for fluid communication with the reservoir and comprising a dilution portion for preparing a sample to be analyzed with a measurement device, the dilution portion being arranged in the channel system so that the sample is transferred from the dilution portion to the measurement device by the fluid medium, wherein the dilution portion includes a set of capillary channels arranged to be filled by capillary action to collect an established quantity of the sample to be diluted by the fluid medium; and a pump operable to transfer the fluid medium from the reservoir to the channel system, the pump comprising at least one plunger, a seal separating the reservoir from the channel system and a delivery system of potential energy configured to provide a repeatable level of the potential energy to transfer the fluid medium from the reservoir to the channel system, wherein the delivery system comprises at least one compressible element to actuate the transfer of the repeatable level of the potential energy.

The invention addresses the shortcomings of existing systems. Particularly the device is suitable for home use without specific experience of the device and education. According to one embodiment of the invention the whole blood has been filtered before performing the dilution. This embodiment yields several advantages. First, it produces a certain constant volume of plasma or other part of the whole sample for the test, i.e. not whole blood. Second, the filtering process takes place separately from the dilution process. Consequently, the movement of the dilution fluid doesn't affect the filtering of the blood. In particular, carrying out the plasma dilution after blood filtration eliminates the effect of hematocrit variation on plasma/buffer dilution ratio.

More particularly, according to one embodiment, in the case of plasma, the order of the operations in the sample handling device is:
filtering of the whole blood to produce plasma (i.e. the sample to be analysed),
metered collection i.e. measurement of the amount of plasma, and
dilution of the plasma and mixing of the plasma with dilution liquid.

According to another embodiment the filtering of the whole sample is an optional procedure. The sample meant to be analysed by the measurement device may also comprise whole blood, too, or any other possible fluids which need to be analysed, filtered or without filtering.

In both embodiments, these particular functions may be achieved by a channel system that includes a preparation portion for a sample in the channel system. The preparation portion may include as functions collection, metering and mixing of the sample, for example.

For mixing the sample handling device includes a pump. The pump includes a reservoir for a fluid medium arranged in connection with the device. The sample is diluted by the fluid medium in the device. In addition, the fluid medium is also used to move the sample from the sample handling device to the measurement device. The pump is configured to be actuated manually. In other words, any other devices or means are not needed to arrange the flow of fluid medium through the device other than the manually actuated pump.

According to specific embodiments, the preparation portion may include as sub-portion a dilution portion and an optional separation portion. The dilution portion includes as functions: collection, metering and mixing of the sample. According to an embodiment a dilution portion may be located after an optional separation portion. More particularly, the dilution portion may be arranged to be directly under the optional separation portion. Then it is possible to apply gravity in the separation to create a sample to be analyzed and/or to fill the predetermined volume arranged for the sample to be measured in the dilution portion. Thus, in the inventive device disclosed here, a passive technique may be used to produce a defined volume of the sample to be diluted and then analysed with the measurement device.

Furthermore, despite variations in the speed of pump actuation by users, the pump provides the same pressure to the sample, contributing to the reliability and consistency of the resulting measurements. Implantation of both the pump and the dilution portion achieves a sample handling device by which the tests may be made by end-users without specific experience and knowledge about the tests and their performances. So to say, both the pump according to the invention and the implementation of the channel system with the dilution portion according to the invention make the device suitable for home use, for example.

One of the main advantages of the title invention is that owing to the invention the pre-processing of the sample has been automatized for the samples which, for one reason or another, must be diluted precisely before use and/or analysis due to the reason that the sample presumably contains too much analyte to be detected. Additional advantages achieved with the invention will become apparent from the description below.

### BRIEF DESCRIPTION OF THE DRAWINGS

The invention, which is not limited to the embodiments set forth below, is described in more detail by making reference to the appended drawings, in which

FIG. 1 shows an example of the sample handling device according to the invention,

FIG. 2 shows the sample handling device shown in FIG. 1 in an exploded view,

FIG. 3 shows schematically a top view of an example of a preparation portion arranged in the channel system of the sample handling device,

FIG. 4 shows in greater detail an example of the preparation portion shown in FIG. 3 in axonometric view,

FIG. 5 shows a cross-section view of the preparation portion shown in FIGS. 3 and 4, together with a top view of a broken out portion of the cross-section,

FIG. 6 shows in greater detail an example of the preparation portion in another embodiment in axonometric view,

FIGS. 7a-7d show a first example of the delivery pump in different operation stages of the pump,

FIG. 8 shows a second example of the pump,

FIGS. 9a and 9b show a third example of the pump in different operation stages of the pump,

FIGS. 10a-10c show a fourth example of the pump in different operation stages of the pump and

FIG. 11 shows an example of the implementation of the dilution portion from upstream diluent flow channel.

### DETAILED DESCRIPTION OF THE INVENTION

The present invention will now be described more fully with reference to the accompanying drawings, in which preferred embodiments of the invention are shown. This invention, however, may be embodied in many different forms and should not be construed as limited to the embodiments shown. In the drawings, like numbers refer to like elements throughout.

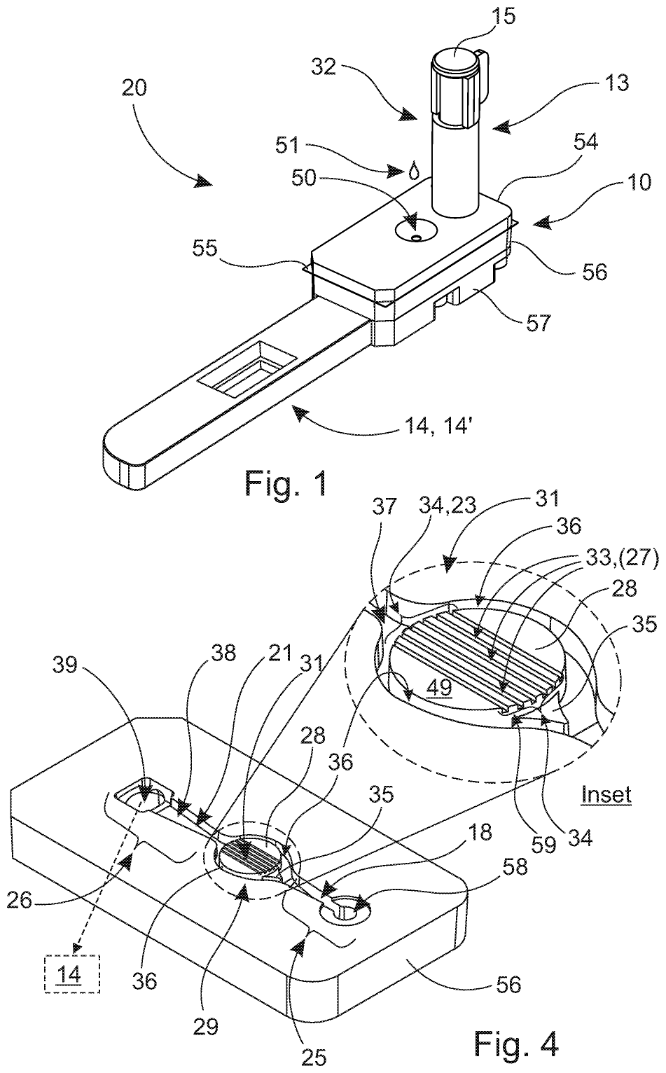

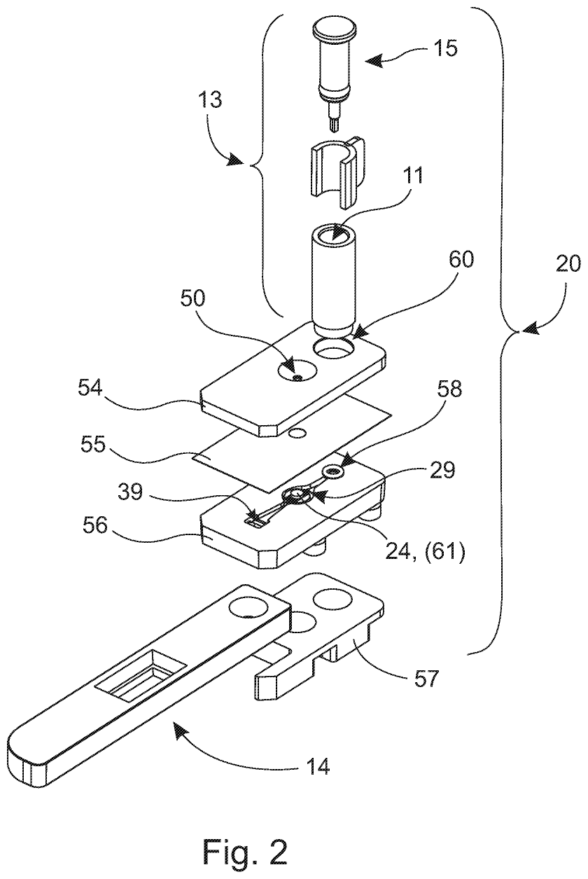

FIGS. 1 and 2 show an example of the sample handling device 10 according to the invention. In FIG. 1 the device 10 is disclosed as assembled and in FIG. 2 the sample handling device 10 shown in FIG. 1 is disclosed in an exploded view.

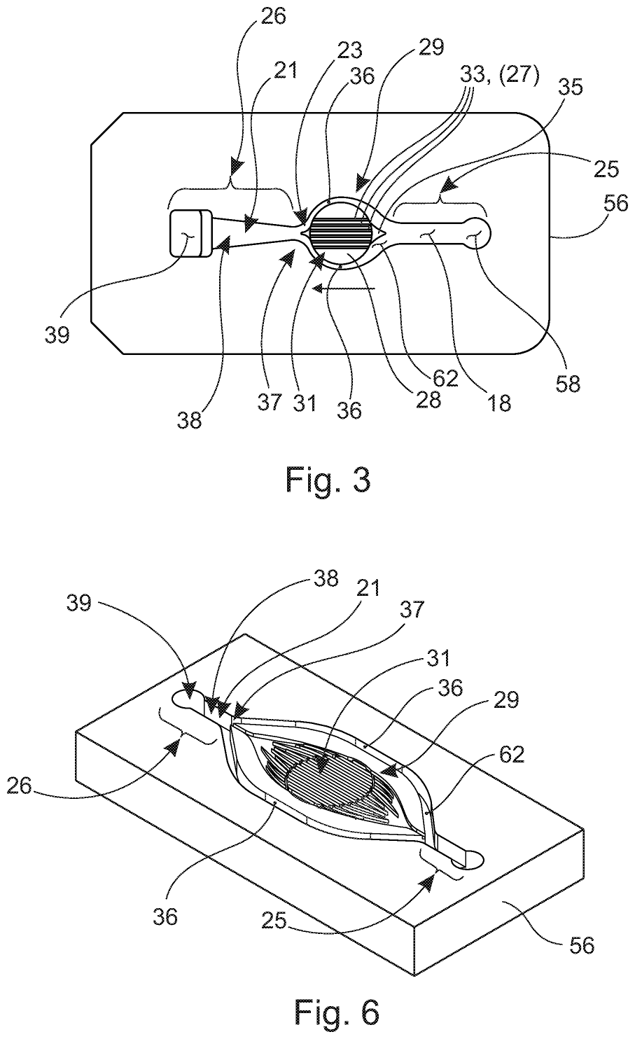

In the embodiment disclosed here, the basic parts of the sample handling device 10 are an assembly 20 with a channel system 21 (FIGS. 3 and 4), a reservoir 11 for holding a fluid medium 12, a pump 13 and a preparation portion 29 for receiving and preparing the sample 19 to be analysed. The preparation portion is also referred to herein as a chamber or, in general, a chamber 62 arranged in connection with the channel system 21. The sample meant to be analyzed may be, for example, plasma. An assembled device 10 is a compact entity to which can be attached and/or integrated with a test and/or a measurement device 14 to perform the analysis of the sample. The sample handling device 10 may have one or more interfaces for this device 14. The measurement system 14 to which the diluted plasma is to be delivered could be a lateral flow measurement system or one of many other types of sensors or detectors. From FIG. 2 it can be seen that the components of the device 10 may be comprised of plate-like elements 54-57 in which a design has been machined and/or molded to achieve the desired function. The elements may be separately described as, for example, a top plate 54, a gasket 55, a base plate 56 and an optional lower plate 57. The components 54-57 may be layered and attached to each other, for example, by mechanical, adhesive and/or other kinds of attachment or joining techniques well-known to persons of skill in the art. These include, for example, screws, holes and nuts to hold the plates together. The layer arrangement of the components makes the device 10 easy and simple to assemble and thus to manufacture.

The device 10 includes a sample receiving portion 50 (blood collection point) into which the whole sample 51, such as a drop of blood, may be placed. In this embodiment, the pump 13 of the device 10 may contain a diluent, such as, for example, a buffer or any other suitable solution for the sample 19. The pump 13 may be perpendicular to the body of the device 10 and to the channel system 21 inside the device 10, as shown in FIG. 1. The sample receiving portion 50 and the pump 13 may be part of or attached to the top plate 54. For this purpose the top plate 54 and also the end of the pump 13 may have an attachment arrangement 60 to attach the pump 13 to the top plate 54.

The pump 13 is arranged in connection with the reservoir 11. The pump 13 is also referred to as the delivery system. The pump 13 is used to transfer the fluid medium 12 from the reservoir 11, or more generally, a reservoir space arranged for the fluid medium 12 to the measurement device 14 through the channel system 21. In the described embodiment the pump 13 includes at least one plunger element 15 or entity to transfer the fluid medium 12 from the reservoir 11 to the channel system 21. The operating principle of the plunger 15 is to reduce the volume of the space of the reservoir 11 to force the fluid medium 12 from the reservoir 11 to the channel system 21 and from there to the measurement device 14. In addition, the pump 13 also includes a seal element 16 arranged to separate the reservoir 11 from the channel system 21, i.e. to keep the fluid medium 12 contained within the pump 13 before the pump 13 is operated. The seal element 16 is in the entrance of the channel system 21, i.e., on the opposite side of the reservoir 11 relative to the plunger 15. In the embodiments shown here, the pump 13 is arranged to release the fluid 12 into the diluent flow channel 18, in response to manual actuation. For this purpose the channel system 21, more particularly, its delivery portion 25 includes buffer entry 58 to which the pump 13 is arranged to feed the fluid medium 12.

In the disclosed embodiment the assembly 20 includes a lateral and elongated channel system 21 (FIGS. 3 and 4) having in the disclosed embodiment three main portions and successive portions forming a passageway. These are the delivery portion 25, the preparation portion 29 in the form of chamber 62 and the output portion 26. The channel system 21 in which the fluid medium 12 is arranged to flow may be mainly straight, i.e., without junctions, in which, for example two different channels, for example being in a perpendicular or other considerable angular arrangement, would join together.

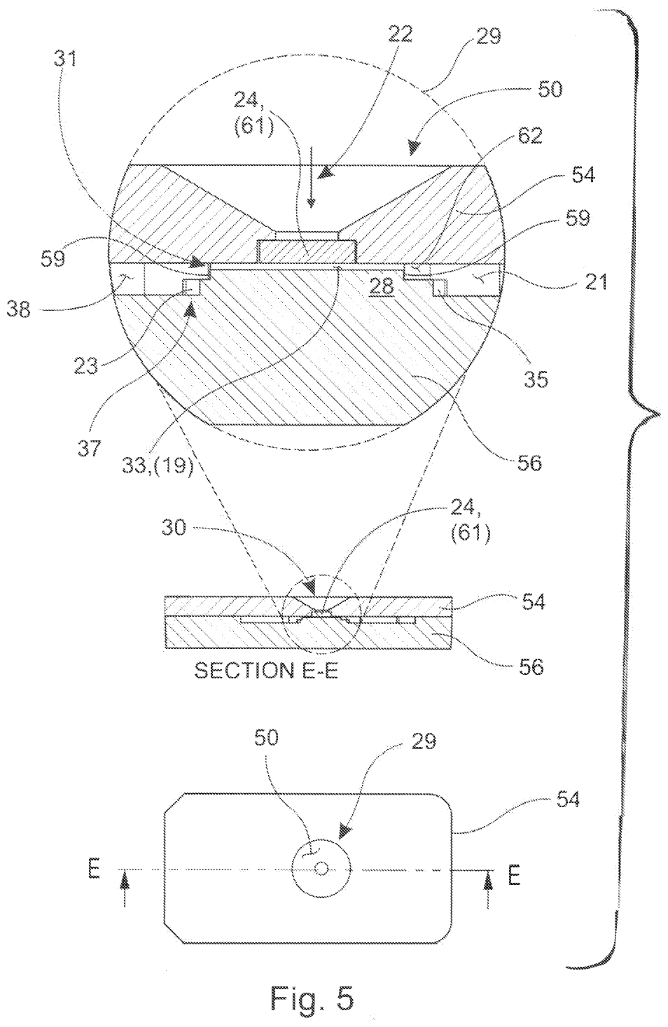

The preparation portion 29 is subdivided in the disclosed embodiment into two portion: an optional separation portion 30 (see FIG. 5, Section E-E) and the dilution portion 31. The channel system 21 is designed to deliver the sample 19 to the measurement device 14 and also dilute the sample 19 as needed for the test to be performed. The plate components 54, 56 form the body of the device 10 and may also form the housing for the channel system 21.

The first portion of the channel system 21, the delivery portion 25, comprises the diluent flow channel 18 and buffer entry 58. The entry 58 is connected to the reservoir 11, which is, in the described embodiment, part of the assembly 20. The reservoir 11 a space which is arranged to store the required volume of fluid medium 12, such as diluent. So, the channel system 21 is in connection with the reservoir 11.

The second portion of the channel system 21 is the preparation portion 29. FIG. 3 shows schematically a top view of an example of a preparation portion 29 arranged in the channel system 21 of the sample handling device 10. FIG. 4 shows in greater detail an axonometric view of an example of the preparation portion 29 shown in FIG. 3. The preparation portion 29 is arranged in the channel system 21 after the seal element 16 of the pump 13. The preparation portion 29 is designed to create, i.e., prepare the sample 19 to be analysed with the measurement device 14. The sample 19 to be analysed is arranged to be transferred from the preparation portion 29 to the measurement device 14 by the fluid medium 12. The connection or entry to the measurement device 14 is on the downstream portion of the channel system 21 relative to the reservoir 11. In other words, the preparation portion 29 is now located in the channel system 21 between the first portion, i.e., diluent flow channel 18 connected to the reservoir 11 and the third main portion 38 of the channel system 21. The third portion 38 of the channel system 21 is the output portion 26, which includes the connection/entry 39 to the measurement device 14.

The seal element 16 separates the channel system 21, more particularly, the preparation portion 29 from the reservoir 11 and the pump 13. Consequently, the sample 19 to be analysed is isolated from the pump 13 and the forces that move the fluid medium 12 from the reservoir 11 to the channel system 21. The advantage of this is that there is no need to dilute the sample to force that through the possible filter 24.

FIG. 5 shows a cross-sectional view of the preparation portion 29 shown in FIGS. 3 and 4. The preparation portion 29 comprises two subparts: a separation portion 30 and a dilution portion 31. Separation portion 30 is used to separate from a whole blood sample 51 that part of the sample 19 to be analysed with a measurement device 14, to which the sample 19 separated from the whole sample 51 is arranged to be transferred by the fluid medium 12. The separation portion 30 separates the plasma from the whole blood 51, so that the red blood cells do not affect the test. Separation portion 30 includes a means to separate plasma from whole blood, shown here as filter material 24, but which could be other means of filtering, too.

The dilution portion 31 is shown on the inset of FIG. 4 and on FIGS. 3, 6 and 11. The dilution portion 31 is designed in the channel system 21 to receive the product of the separation portion 30, i.e. the plasma that has been separated by filter 24. The dilution portion 31 is arranged to dilute the sample 19 to be analysed, i.e. the plasma, by the fluid medium 12, i.e. diluent, to obtain a volume and concentration suitable for the measurement. In addition, the dilution portion 31 is also designed to collect a certain relatively precise quantity of the sample 19 to be diluted and analysed with the measurement device 14 so that a quantitative measurement of the sample 19 can be made. The dilution portion 31 thus has also both a collection function and a metering function in the same component by means of which the dilution has been made.

The dilution portion 31 includes a set of channels 27, more particularly, collection channels 33, also referred to herein as capillary channels. In the flow direction 22 of the sample 19 in the preparation portion 29, the dilution portion 31 is arranged after the separation portion 30. More particularly, the dilution portion 31, i.e. the collection channels 33, are arranged directly under plasma separation filter 24 in separation portion 30. The collection channels 33 are arranged to fill by capillary action from the separation portion 30, i.e. from the filtering means 24. In addition, capillary breaks 34 are found at the ends of the set of channels 27. Thus, the collection channels 33 are designed to fill to a volume fixed by capillary breaks 34 at the ends of the channels 33 producing an established, i.e., known quantity of the sample 19 to be diluted and then analyzed. In other words, when the collection channels 33 are full, the flow of the sample 19 from the sample receiving portion 50 ends to the dilution portion 31. Thus, the volume of the sample 19 in the dilution portion 31 is precisely defined and known.

The dilution portion 31 includes in the disclosed embodiment a body 28 arranged in the area of the channel system 21 and the base plate 56. The body 28 is formed in the chamber 62 of the channel system 21 arranged in connection with the dilution portion 31. The body 28 extends from the base plate 56 in the direction perpendicular to the elongated direction of the channel system 21. The upper surface 49 of the body 28 is on the level of the lower surface of the top plate 54. To the upper surface 49 of the body 28 has been arranged the collection channels 33. The collection channels 33 are in the parallel direction relative to the elongated direction of the channel system 21. The collection channels 33 may be, for example, micro-machined to the upper surface 49 of the body 28.

In one embodiment the collection channels 33 have a total volume of, for example, 1.4 μl. In general, the total volume of the collection channels 33 can be, for example, 0.5-5 μl. This is the volume of plasma, i.e., sample 19 to be metered. Now there are six collection channels 33 (slots) in the set of channels 27. Each collection channel 33 is 0.2 mm deep and 0.2 mm wide. The diameter of the chamber 62 may be, for example, 5-10 mm, such as, for example, 6 mm. The rules for the dimensioning the collection channels 33 comes from capillary forces. Particularly, the ends of the collection channels 33 are designed with capillary breaks 34 where they meet the upstream and downstream diluent flow channels 18, 38. The ends of the set of channels 27 are open to the chamber 62 of channel system 21 arranged in connection with the dilution portion 31. The volume of the chamber 62 is relatively large and, as known, owing to the capillary forces don't draw liquids beyond this kind of break 34. The set of channels 27 are arranged in the channel system 21 so that at least part of the fluid medium 12 is arranged to flow through the set of channels 27 in order to flush the sample 19 from the collection channels 33. The cross-section profile of the collection channels 33 may be, for example, square, circular or triangle. In pilot stage tests of the device it was noticed that the square profiled grooves, i.e., channels 33 were the fastest filling, for example, in the case of plasma.

The channel system 21 includes an upstream diluent flow channel 18 arranged to direct a flow of fluid medium 12 from the pump 13 to the upstream ends of the set of channels 27 of the dilution portion 31. In addition, the dilution portion 31 is also arranged to split the flow of fluid medium 12 in the channel system 21. Splitting is accomplished by means of a flow splitter 35 arranged to the dilution portion 31. The splitter 35 is in the body 28 on the side of the bottom plate 56. The flow splitter 35 directs most of the flow of fluid medium 12 to the side channels 36 on opposite sides of the dilution portion 31. Thus, only a portion of the fluid medium 12 is arranged to flow into the set of channels 27 of the dilution portion 31. In addition, the flow of fluid medium 12 in the channel system 21 takes place perpendicularly to the flow direction 22 of the sample 19 from the sample receiving portion 50. This division of the flow may be achieved by suitable shaping of the diluent flow channel 18 and/or the flow splitter 35. The cross section of the upstream diluent flow channel 18 is configured to widen towards the chamber 62, i.e., diluent portion 31.

More particularly, the channel system 21 includes an upstream diluent flow channel 18 arranged to guide a flow of the fluid medium 12 to, around, from and through the set of channels 27. More generally, the dilution portion 31 includes an arrangement 23 to mix the sample 19 being in the set of channels 27 having a predefined quantity and the fluid medium 12. In addition, for this particular mixing purpose at the downstream end of the dilution portion 31, particularly, in a downstream end of the side channels 36, i.e. before the downstream diluent flow channel 38, the side channels 36 merge at a convergence portion 37 included in the dilution portion 31 which turns the flow of fluid medium 12. The convergence of the fluid flows generates pressures that draw the plasma or, more generally, sample 19 out of the collection channels 33 of the set of channels 27. The converge portion 37 is in the body 28 on the side of the bottom plate 56. Between the collection channels 33 and the splitter 35 and converge portion 37, there may still be a step 59 at both ends of the collection channels 33.

Side channels 36 may be designed to squeeze fluid medium 12, increasing velocity and lowering fluid pressure. The downstream part of the diluent flow channel 38 is arranged to taper so that when the fluid medium 12 discharges to atmospheric pressure, the pressure at the convergence portion 37 is such as to cause the plasma to flow from the channels 33 and also mix with the diluent fluid 12. Mixing takes place in accordance with the Bernoulli principle. In that the flow velocity will be increased and the pressure will drop. The pressure at the convergence portion 37 is then sufficiently low. The downstream part of the diluent flow channel 38 is designed to control the flow speeds and pressures so that it draws plasma from the collection channels 33 while the upstream diluent flow channel 18 simultaneously replaces the plasma without causing any net flow through the separation filter/membrane 24. In addition, the downstream part of the diluent flow channel 38 and the convergence portion 37 is designed to prevent back flow of diluted sample towards the dilution portion 31. Geometry and dimensioning of the channel system 21, chamber 62 and the dilution portion 31 are configured to achieve mixing based on the Bernoulli principle. Also, the channel system 21 which is arranged to have a variable cross-sectional area in connection with the dilution portion 31 plays a role causing the desired effects.

In use, the dilution portion 31 is initially air-filled and open to atmospheric pressure. When whole blood 51 is put on the receiving section 50, i.e. on top of the plasma separation filter 24, the plasma passively filters through and fills the collection channels 33 by passive capillary action, up to the capillary breaks or stops 34. Filled collection channels 33 will hold a metered amount of plasma. The capillary breaks 34 at both ends of each channel 33 together prevent over filling of plasma. It is preferred that the capillary breaks 34 are on a circular locus under the edge of a circular filter material 24. Circular ends may be achieved by the dilution portion 31, more particularly, body 28 having a circular form factor. Due to the circular form factor the length of collection channels 33 in the middle of the chamber 62 and, thus, of the channel system 21 is the greatest and the length of the collection channels 33 diminishes towards the both sides of the body 28 and thus of the dilution portion 31. The dilution portion 31 then requires a suitable volume of diluent 12 to be passed through it at a suitable flow rate. When this is done, by the delivery system, such as pump 13 or by some other diluent flow control system, then the plasma is mixed with and diluted by the diluent 12 in a reproducible ratio, and the diluted plasma is delivered at the diluted plasma exit point 39 being in the downstream diluent flow channel 38. This embodiment is thus able to collect 1.4 μl of plasma, dilute it 1:100 with diluent 12 and deliver 100 μl of diluted plasma. Thus, by means of the device 10 it is possible to mix a relatively small amount of higher viscosity (sample 19) and a relatively large amount of lower viscosity (diluent 12) liquids together very effectively and reproducible.

In alternative embodiments of the dilution portion 31 different dimensions may be used to get different metered volumes, different mix ratios and different delivered volumes. One skilled in the art also realizes that other cross-sections and shapes may be used for the collection channels 33 and diluent flow channels, while maintaining appropriate geometries at the flow splitter 35 (or more generally, flow separation feature) and the convergence portion 37 (more generally, flow convergent feature) to ensure the required interactions between the diluent (fluid medium 12) and the plasma (sample 19).

According to one embodiment, combined with the already previously described “dilution system”, the device 10 also includes an arrangement to provide a controlled flow of an aliquot of fluid, such as diluent, or more generally, a fluid medium 12, to the “dilution system”, i.e. to the dilution portion 31 of the device 10 and on to the measurement system 14 with the diluted plasma, or the sample. This particular portion of the device 10 may be termed the “delivery system” 32.

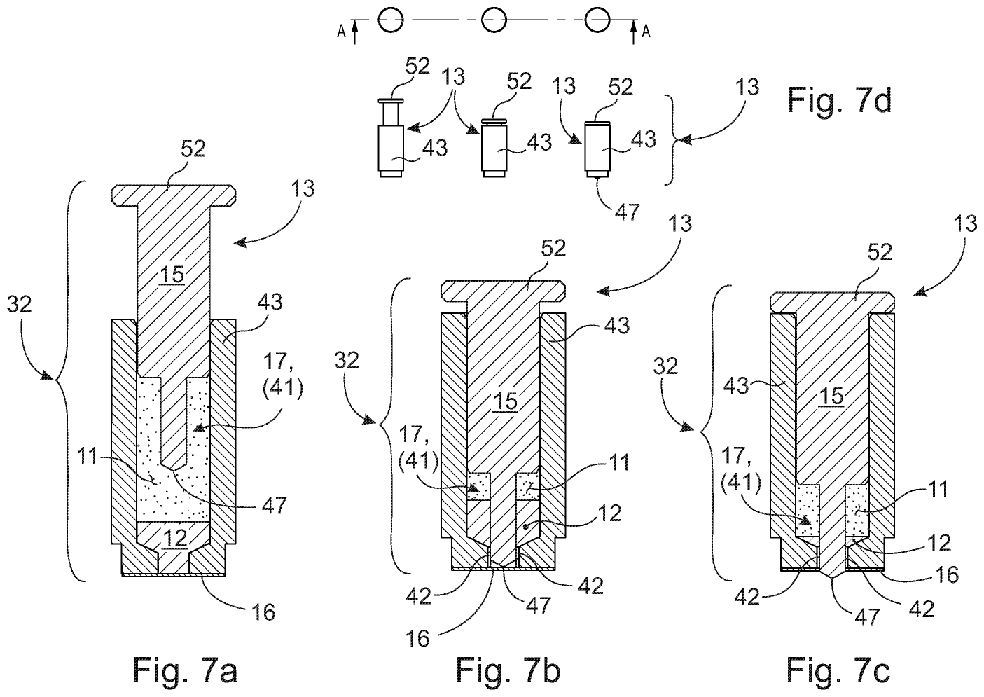

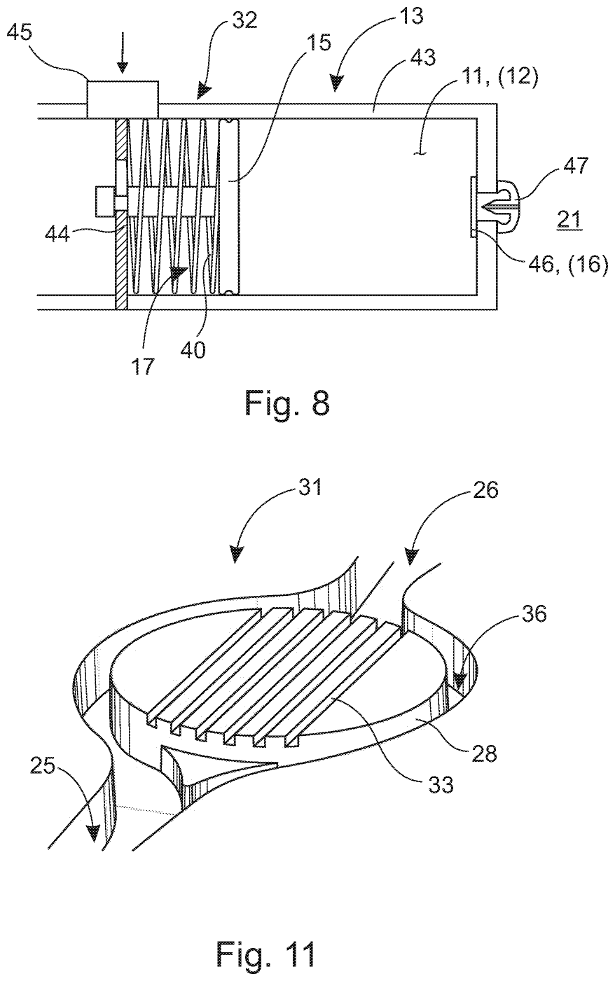

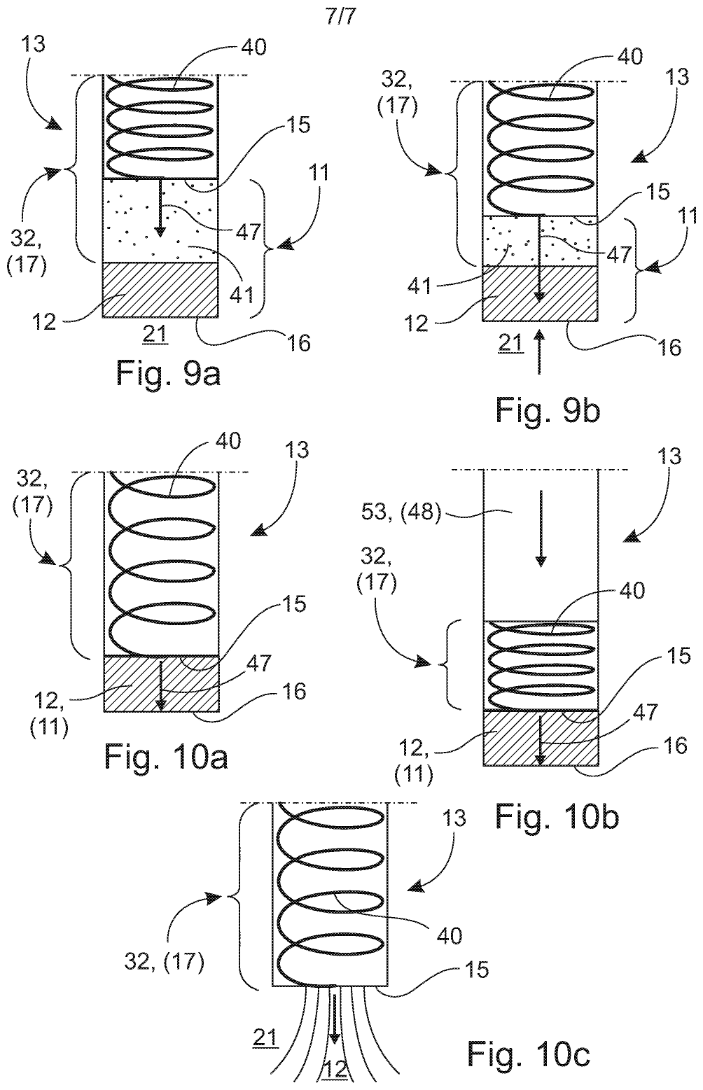

FIGS. 7-10 show alternate embodiments relating to the “delivery system” including, for example, pump 13. As shown here, the pump 13 includes at least one plunger 15 and may be equipped with some source of potential energy to provide repeatable transfer of the fluid medium 12 from the reservoir 11 to the channel system 21 of the device 10. Delivery system 32 includes means to pressurize or propel the fluid medium 12 so that when it is released from the reservoir 11 it flows in a repeatable, controlled manner through the device 10, independent of the speed or force of manual actuation which has been used to release the fluid medium 12 from the reservoir 11. Delivery system 32 may include one or more compressible elements 17 arranged in the pump 13. The compressible elements 17 may be in the reservoir 11 between the plunger 15 and the fluid medium 12 (FIG. 7) or also outside of the reservoir 11, behind the plunger 15 (FIG. 8) or in both positions (FIG. 9).

A first embodiment of pump 13 is shown in FIGS. 7a-7c in different stages of the operation. In FIG. 7d there is disclosed a front view of the pump 13 showing the relationship between the plunger 15 and housing. In this embodiment, the fluid medium 12 is propelled by air pressure, which is developed by manual depression of a plunger 15. The fluid medium 12 is contained in a tank 43 forming a reservoir 11 in which there is both the fluid medium 12 together with a volume of air 41. The air 41 serves as the compressible element 17. At a manufacturing stage of the pump 13 and/or device 10, the air 41 may be at atmospheric pressure and the access from the reservoir 11 to the channel system 21 is sealed by a pierceable film, i.e., a seal element 16. The volume of fluid medium 12 is set during manufacture and it is incompressible. Prior to use the air 41 occupies a maximum volume (V₁) at a minimum pressure (P₁). When the plasma in the dilution portion 31 is ready to be diluted and delivered, the user is prompted to depress the plunger 15 (FIG. 7a). Depression of the plunger 15 pressurizes the compressible air 41 in the reservoir 11 (FIG. 7b) and the consequence is that the air 41 is compressed to a reduced volume (V₂) at an increased pressure (P₂). The speed and force with which the user depresses the plunger 15 has minimal effect on what pressure P₂ is achieved in the air 41, so the natural variability on how users push the plunger 15 has no significant effect on the test. The last part of the travel of the plunger 15 pierces the film seal 16 and also latches the plunger 15 down, so that it does not move when the user stops pressing (FIG. 7c). In addition, when the seal element 16 has been pierced the plunger 15 is immediately stopped from moving although the user may still be pressing it downwards. The stopping mechanism may be arranged at the end of the plunger 15. For example, the widening 52 at the end of the plunger 15 may come into contact with the tank 43, i.e., the body of the pump 13, to stop the movement of the plunger 15. At the point of piercing the seal 16 the maximum system pressure P₂ is reached. When the seal 16 is pierced the fluid medium 12 is released and it flows out of the reservoir space 11, propelled only by the pressurised air 41. As the fluid medium 12 flows out from the reservoir 11, the air space between the end of the plunger 15 and the surface of the fluid medium 12 expands and the pressure reduces in a predictable repeatable way. The size of the outlet channel 42 to the channel system 21, the air pressure and the downstream back pressure combine to control the flow rate of the fluid medium 12. The result is that fluid medium 12 flows through the “dilution system”, i.e. dilution portion 31, at a known flow-rate which gives the correct dilution independent of whether the manual actuation of the plunger 15 was done slowly or quickly and how hard it was pressed. The fluid medium 12 is chased by the pressurised air 41, so depending on how much air 41 is used, all of the fluid medium 12 may be flushed through the “dilution system” or some fluid medium may be left in the “dilution system”.

This first embodiment shown in FIGS. 7a-7d has been demonstrated in pilot tests of the device 10 to work well with a reservoir 11 containing 150 μl of fluid medium 12 and 490 μl of unpressurised air 41 (1:3,3). The reservoir 11 had a bore of 6.7 mm and a plunger 15 stroke of 12.6 mm. The air gauge pressure reached 3.1 bar before piercing the seal element 16 and reduced to 0.3 bar when all the fluid medium 12 was expelled. The fluid medium 12 took 0.5 seconds to flow through the “dilution system” and to mix with and dilute the plasma that was waiting in the dilution portion 31. More generalized, the volume ratio of fluid medium 12 and unpressurised air 41 may be, for example, 1:2 to 1:4. The reservoir 11 may have a bore of 4-8 mm and a plunger 15 stroke of 8-15 mm. The air gauge pressure may be 2-4 bar before piercing the seal element 16.

In this first described embodiment the user does a fixed quantity of work (=force×distance) in a variable amount of time, to compress the air 41 and store a certain amount of potential energy in the compressed air 41. When the seal 16 is pierced, the potential energy in the air 41 is converted to the kinetic energy of the flowing fluid medium 12, at a rate controlled by the pressures and flow channel geometries, independent of what the user did.

A second embodiment of this “delivery system” is disclosed in FIG. 8 which shows the pump 13 in the loaded stage, i.e. before it is used. In this embodiment, the fluid medium 12 is propelled by pressure from a pre-loaded spring 40 which serves as the compressible element 17 of the delivery system 32 in the pump 13.

This second embodiment normally leaves some fluid medium 12 in the dilution portion 31 after the operation of the pump 13 and also in the device 10, but this has no detrimental effect on the test results. In other words, the pump 13 is configured to provide a burst of the fluid medium 12 from the reservoir 11 to the dilution portion to transfer the sample 19 to the measurement device 14. The fluid medium 12 is again contained in a tank 43 forming the reservoir 11 for the fluid medium 12 with a sprung plunger 15 held in compression by a catch 44. When the fluid medium 12 is required, the user pushes a button 45 which releases the spring catch 44. The spring 40 then applies force to the plunger 15 and pressurises the fluid medium 12 which is in the reservoir 11. In other words, the delivery system 32 now includes a source of potential energy, such as, for example, a spring element 40 arranged to affect the plunger 15. Some other mechanical element/system than spring 40 may be possible. The pressure in the fluid medium 12 deflects the film seal 46 and pierces it on the spike 47. The fluid medium 12 then flows to the channel system 21 and through the dilution portion 31 at a controlled rate. Another version of this embodiment may pierce the film seal 46 directly with a pin operated by the push button 45 (not shown). One particular advantage of this spring embodiment is that the fluid medium 12 is not exposed to air so there is no possibility of foaming or of air mixing with the fluid medium 12.

In this second described embodiment a certain amount of potential energy is stored in the compressed spring 40 at manufacturing time. When the seal 46 is pierced and the spring catch 44 released, the potential energy in the spring 40 is converted to the kinetic energy of the flowing fluid medium 12, at a rate controlled by the spring force and flow channel geometries, independent of what the user did.

FIGS. 9a and 9b show a third example of the pump 13 in different stages of operation. In this embodiment there are two compressible elements 17. Those are now both a pre-loaded spring element 40 and also a volume of compressible air or other gas or fluid 41 arranged in the pump 13 with the spring element 40. The initial pressure of the compressible air, gas or fluid 41 may be defined to be P₁ (FIG. 9a). The pump 13 includes again a releasing mechanism (not shown) by means of which the compressed spring 40 may be released. At the end of the spring element 40 there is a plunger 15 acting on the air 41 which is in the reservoir space 11. Releasing of the spring 40 causes the air pressure to rise from P₁ to Pz. A spike or corresponding piercing element 47 may again be integrated in the pump 13, for example on the plunger 15, to pierce the seal foil 16 at the entrance to the channel system 21 and thus release the fluid medium 12 at pressure Pz from the reservoir space 11 to the channel system 21 (FIG. 9b). The pressure then becomes P₁ again. One advantage of this third embodiment is that all the fluid medium 12 can be flushed through the dilution portion 31 of the device 10 by the air, gas or fluid 41.

FIGS. 10a-10c show a fourth example of the pump 13 in different stages of operation. In this embodiment the basic operating principle is similar to the second embodiment previously described in FIG. 8. Instead of using a preloaded compressible element 17, for example a spring, in this embodiment the compressible element 17 is loaded by a movable element 48. Specifically, the compressible element 17 is compressed, i.e. loaded, by the movement of a piston element 48, such as a piston rod 53 (FIG. 10b), which can be pressed starting from the initial position presented in FIG. 10a, for example, by a hand. Again, the component arranged at an end of the spring 40 acts as a plunger 15. In the described delivery system 32 the spring 40 compresses but the fluid medium 12 in the reservoir space 11 doesn't compress. For example, at the end of the plunger 15 may again be a spike or corresponding piercing element which pierces the seal 16 and releases the fluid medium 12 to the channel system 21 (FIG. 10c). One advantage of this fourth embodiment is that the compressible element 17 is not compressed during storage between manufacture and use, so ageing effects such as creep are reduced.

In the reservoir 11 of the pump 13 there will be a certain relatively precise volume of fluid medium 12 and a certain relatively precise volume of air 41 (or some gas or fluid—or some mechanical element, such as, a spring implemented reproducible way). In the device 10 according to the invention the finger movement of the user is standardized by using dimensions of the plunger 15 and cylinder which are reproducibly manufactured and the amounts of fluid medium 12 and air 41 (or gas/spring) dosed to the pump 13 for the device 10 in factory. Owing to these the fluid medium 12 flows always through the dilution portion 31 in the same repeatable way (more precisely, at about the same speed).

Skilled persons will recognize that the “dilution system”, i.e. the dilution portion 31 embodiments having the features described above, could be used in alternative applications with other means of delivering diluent, such as a syringe pump in an automated instrument. Similarly, the “delivery system”, i.e. the pump 13 embodiments having the features described above, could be used to provide a controlled flow of fluid from manual actuation, as known in other fields of use. Thus, the pump 13 may be a separate entity. However, together the dilution portion 31 and the pump 13 of the device 10 according to the invention operate with great synergistic effects, since those both make the device 10 suitable for use by an unexperienced user who is not familiar with the measures of preparation of the sample for the analysis. In other words, it is not possible to implement the device 10 without both entities. As generally known, such a knowledge can't be obtained from the end-users of home tests made by common people without specific education. In operation, sample 51 to be analysed is inserted in the sample receiving portion 50, and after a period of time during which the dilution portion 31 is filled, the pump 13 is manually launched which provides a constant flow of diluent independently of how it was launched (quickly or slowly). Owing to the invention, a constant speed of the diluent 12 via the channel system 21 and also the dilution portion 31 (i.e., flushing of the sample 19 from capillaries 33 and also mixing to the diluent 12) are achieved.

In addition, the sample handling device 10 may be implemented even without the separation portion 30, too. In that case, for example, the sample to be analyzed is formed somewhere else and thereafter brought to the device 10 to drop to the sample into the sample receiving portion 50. And of course, the whole blood 51 may also be analysed. In that case the sample may be any kind of liquid, fluid, emulsion or suspension, i.e., not only (a part of) blood. In the case of blood, the sample can also be, in addition to plasma, for example, also serum.

In other words, in general, the device 10 includes a sample receiving portion 50 arranged in connection with the dilution portion 31. The sample receiving portion 50 is arranged to close the set of channels 27 towards the sample receiving portion 50 by an element 61 preventing back flow. The element 61 can be, according to a first embodiment, the filter 24 arranged to separate part of the whole sample 51 to be measured but it can also be a permeable or semipermeable membrane through which the sample 19 to be analysed passes essentially without affecting the sample. Thus, in the latter situation, loose filter material may be applied so that nothing will be filtered out. This element 61 thus closes the elongated side of the set of channels 27 upwards and thus acts as kind of a roof for the collection channels 33, preventing those channels from flooding on the one hand, and on the other hand, preventing the fluid medium from flushing the sample 19 from the capillaries and penetrating essentially towards filter 24 or membrane. The filter material or corresponding element 61 is immediately in contact with the capillaries into which the filtered sample 19 penetrates from the filter or corresponding element 61 driven by capillary forces. The capillaries are directly in physical contact with the filter material or corresponding element 61 and owing to this it will not be possible to collect between the capillaries and the filter material considerable amounts of the sample 19 to be analysed, rather that is in the capillaries from which it is flushed by the fluid medium 12. In other words, the collection channels 33 have been filled from their elongated side that is open upwards, i.e., towards to the sample receiving portion 50. So to say, the channels may also be called slots or grooves. Thus, the collection channels 33 are very precisely filled with a relatively constant amount of sample 19 to be tested, resulting in a measured dilution which is very critical for the analysis. Without a very precise and constant amount of sample 19, the dilution of the sample 19 is not accurate.

Several different advantages have been achieved by the invention. In the device 10 according to the invention the preparation portion 29, particularly the dilution portion 31, and the pump 13 may be combined and manufactured as disposable components and at low cost. The plasma separation filter 24, more particularly, separation portion 30 may be integrated with the other parts, particularly, with the dilution portion 31 and arranged so that a drop of blood 51, for example, can easily be put on the filter 24, or more generally, the separation portion 30. All the diluted plasma can be turned through 90° and applied to a lateral flow test. The whole test system can be made as a single-use, disposable test, suitable for untrained users to use at home. The combination of the “dilution system” and “delivery system” can be used as a stand-alone sample preparation device or as an integrated part of a complete measurement system.

At least one sample handling device 10 can be part of the measurement device 14. According to one embodiment the measurement device 14 is a lateral flow test device 14′. In those the sample 19 reacts with the labelled reagents in a known manner. The lateral flow test device may then be still inserted to a reader device which provides the quantitative result for the test.

One further aspect of the invention is the use of the sample handling device 10 in laboratory analysis, point of care testing, point of need testing, field analysis and home testing. The device 10 is particularly advantageous in home testing since the common person is not required to be able to pipet. The device 10 according to the invention is very suitable for mass-production, it is easy-to-use and cheap to manufacture.

While the present invention has been disclosed with reference to certain embodiments, numerous modifications, alterations and changes to the described embodiments are possible without departing from the concept of the invention as defined in the appended claims.

1-25. (canceled)

26. A sample handling device comprising:
a reservoir to hold a fluid medium;
a channel system arranged for fluid communication with the reservoir and comprising a dilution portion for preparing a sample to be analyzed with a measurement device, the dilution portion being arranged in the channel system so that the sample is transferred from the dilution portion to the measurement device by the fluid medium, wherein the dilution portion includes a set of capillary channels arranged to be filled by capillary action to collect an established quantity of the sample to be diluted by the fluid medium; and
a pump operable to transfer the fluid medium from the reservoir to the channel system, the pump comprising at least one plunger, a seal separating the reservoir from the channel system and a delivery system of potential energy configured to provide a repeatable level of the potential energy to transfer the fluid medium from the reservoir to the channel system, wherein the delivery system comprises at least one compressible element to provide the repeatable level of the potential energy.

27. The sample handling device according to claim 26, wherein the channel system further comprises: a sample receiving portion arranged for fluid communication with the dilution portion; and an element to close the set of capillary channels towards the sample receiving portion to prevent back flow.

28. The sample handling device according to claim 27, further comprising a separation portion between the sample receiving portion and the dilution portion, wherein the separation portion constitutes the element to prevent back flow and is constructed to separate the sample to be analyzed with the measurement device from a whole sample received by the sample receiving portion.

29. The sample handling device according to claim 27, wherein the dilution portion is located after the sample receiving portion in a flow direction of the sample to be analyzed with the measurement device.

30. The sample handling device according to claim 27, wherein the dilution portion is arranged to be directly under the sample receiving portion.

31. The sample handling device according to claim 26, further comprising capillary breaks at ends of the set of capillary channels.

32. The sample handling device according to claim 26, wherein the channel system includes a chamber and the set of capillary channels are in the chamber such that the ends of the set of capillary channels open into the chamber.

33. The sample handling device according to claim 26, wherein the channel system is arranged to have a variable cross-section area in connection with the dilution portion.

34. The sample handling device according to claim 27, wherein the set of capillary channels include an upwardly facing, elongated side that is arranged to be open towards the sample receiving portion, and the set of capillary channels is configured to fill from the upwardly facing, elongated side.

35. The sample handling device according to claim 26, wherein the set of capillary channels is arranged in the channel system so that at least part of the fluid medium flows through the set of capillary channels.

36. The sample handling device according to claim 26, wherein the dilution portion is configured to mix the sample in the set of capillary channels with the fluid medium.

37. The sample handling device according to claim 26, further comprising an upstream diluent flow channel arranged in the channel system to guide a flow of the fluid medium around and through the set of capillary channels.

38. The sample handling device according to claim 26, wherein
the channel system further comprises an upstream diluent flow channel for directing a flow of fluid medium to an upstream end of the set of capillary channels; and
the dilution portion includes opposite sides, a flow splitter at the upstream end of the set of capillary channels and a side channel on each opposite side of the dilution portion, wherein the flow splitter is arranged to split the flow of fluid medium to flow most of the fluid medium through the side channels and a minority of the fluid medium into the set of capillary channels.

39. The sample handling device according to claim 38, wherein
the dilution portion further comprises a convergence portion in a downstream end of the side channels for merging the side channels and turning the flow of fluid medium to generate a pressure effect to draw the sample out of the set of capillary channels; and
a downstream part of the diluent flow channel is tapered so that when the fluid medium discharges to atmospheric pressure, the pressure at the convergence portion causes the sample to flow and mix with the fluid medium.

40. The sample handling device according to claim 28, wherein
the channel system further comprises an upstream diluent flow channel for directing a flow of fluid medium to upstream ends of the set of capillary channels; and
the dilution portion includes opposite sides, a flow splitter at the upstream end of the set of capillary channels and a side channels on each opposite side of the dilution portion, wherein the flow splitter is arranged to split the flow of fluid medium to flow most of the fluid medium through the side channels and a minority of the fluid medium into the set of capillary channels.

41. The sample handling device according to claim 40, wherein
the dilution portion further comprises a convergence portion in a downstream end of the side channels for merging the side channels and turning the flow of fluid medium to generate a pressure effect to draw the sample out of the set of capillary channels; and
a downstream part of the diluent flow channel is tapered so that when the fluid medium discharges to atmospheric pressure, the pressure at the convergence portion causes the sample to flow and mix with the fluid medium.

42. The sample handling device according to claim 41, wherein the downstream part of the diluent flow channel is arranged to control the flow speeds and pressures to draw the sample from the set of capillary channels while the upstream diluent flow channel simultaneously is arranged to replace the sample without causing any net flow through the separation portion.

43. The sample handling device according to claim 32, wherein the channel system includes a body comprising a circular form factor arranged in an area of the chamber of the channel system, wherein the set of capillary channels are arranged in the body.

44. The sample handling device according to claim 32, wherein the dilution portion includes opposite sides and a side channel on each opposite side, and a length of the set of capillary channels is configured to be greatest in a middle of the chamber of the channel system and is configured to diminish towards the side channels.

45. The sample handling device according to claim 26, wherein the channel system is arranged to form a mainly straight passageway for the fluid medium.

46. The sample handling device according to claim 26, wherein the compressible element comprises a spring element pre-loaded in the pump or arranged to be loaded by movement of a pusher element.

47. The sample handling device according to claim 26, wherein the compressible element comprises a volume of compressible material arranged with or without the spring element.

48. The sample handling device according to claim 26, wherein the pump is configured to provide a burst of the fluid medium from the reservoir.

49. The sample handling device according to claim 26, wherein the pump is configured to release the fluid medium into the channel system in response to a manual actuation.

50. A combination comprising the sample handling device according to claim 26 and the recited measurement device.

51. The combination according to claim 50, wherein the measurement device comprises a lateral flow test device.

52. A method of performing at least one of a laboratory analysis, a point of care test, a point of need test, a field analysis and a home test of a sample, comprising utilizing the sample handling device according to claim 26.
